SEQUENCE LISTING (9 sequences)

SEQ ID NO: 1 - protein, 384 aa, Mouse Polyomavirus Strain BG
MAPKRKSGVSKCETKCTKACPRPAPVPKLLIKGGMEVLDLVTGPDSVTEIEAFLNPRMGQPPTPESLTEGGQYYGWSRGINLATSDTEDSPENNTLPTWSMAKLQLPMLNEDLTCDTLQMWEAVSVKTEVVGSGSLLDVHGFNKPTDTVNTKGISTPVEGSQYHVFAVGGEPLDLQGLVTDARTKYKEEGVVTIKTITKKDMVNKDQVLNPISKAKLDKDGMYPVEIWHPDPAKNENTRYFGNYTGGTTTPPVLQFTNTLTTVLLDENGVGPLCKGEGLYLSCVDIMGWRVTRNYDVHHWRGLPRYFKITLRKRWVKNPYPMASLISSLFNNMLPQVQGQPMEGENTQVEEVRVYDGTEPVPGDPDMTRYVDRFGKTKTVFPGN

SEQ ID NO: 2 - protein, 372 aa, Hamster Polyomavirus
MCKPLWKPCPKPANVPKLIMRGGVGVLDLVTGEDSITQIEAYLNPRMGQNKPGTGTDGQYYGFSQSIKVNSSLTADEVKANQLPYYSMAKIQLPTLNEDLTCDTLQMWEAVSVKTEVVGVGSLLNVHGYGSRSETKDIGISKPVEGTTYHMFAVGGEPLDLQGLVQNYNANYEAAIVSIKTVTGKAMTSTNQVLDPTAKAKLDKDGRYPIEIWGPDPSKNENSRYYGNFTGGTGTPPVMQFTNTLTTVLLDENGVGPLCKGDGLYLSAADVMGWYIEYNSAGWHWRGLPRYFNVTLRKRWVKNPYPVTSLLASLYNNMLPTIEGQPMEGEAAQVEEVRIYEGTEAVPGDPDVNRFIDKYGQQHTKPPAKPAN

SEQ ID NO: 3 - DNA, 1155 bp, Mouse Polyomavirus Strain BG
atggcccccaaaagaaaaagcggcgtctctaaatgcgagacaaaatgtacaaaggcctgtccaagacccgcacccgttcccaaactgcttattaaagggggtatggaggtgctggaccttgtgacagggccagacagtgtgacagaaatagaagcttttctgaaccccagaatggggcagccacccacccctgaaagcctaacagagggagggcaatactatggttggagcagagggattaatttggctacatcagatacagaggattccccagaaaataatacacttcccacatggagtatggcaaagctccagcttcccatgctcaatgaggacctcacctgtgacaccctacaaatgtgggaggcagtctcagtgaaaaccgaggtggtgggctctggctcactgttagatgtgcatgggttcaacaaacccacagatacagtaaacacaaaaggaatttccactccagtggaaggcagccaatatcatgtgtttgctgtgggcggggaaccgcttgacctccagggacttgtgacagatgccagaacaaaatacaaggaagaaggggtagtaacaatcaaaacaatcacaaagaaggacatggtcaacaaagaccaagtcctgaatccaattagcaaggccaagctggataaggacggaatgtatccagttgaaatctggcatccagatccagcaaaaaatgagaacacaaggtactttggcaattacactggaggcacaacaactccacccgtcctgcagttcacaaacaccctgacaactgtgctcctagatgaaaatggagttgggcccctctgtaaaggagagggcctatacctctcctgtgtagatataatgggctggagagttacaagaaactatgatgtccatcactggagagggcttcccagatatttcaaaatcaccctgagaaaaagatgggtcaaaaatccctatcccatggcctccctcataagttcccttttcaacaacatgctcccccaagtgcagggccaacccatggaaggggagaacacccaggtagaggaggttagagtgtatgatgggactgaacctgtaccgggggaccctgatatgacgcgctatgttgaccgctttggaaaaacaaagactgtatttcctggaaattaa

SEQ ID NO: 4 - DNA, 87 bp, Mouse Polyomavirus Strain BG
  Counter strand of the sequence coding for the BC2-Loop of the VP1-protein of the mouse polyomavirus strain BG
catactccatgtgggaagtgtattattttctggggaatcctctgtatctgatgtagccaaattaatccctctgctccaaccatagta

SEQ ID NO: 5 - DNA, 33 bp, Artificial Sequence
  Primer 080BN093 f
gccannnnnnnaatacacttcccacatggagta

SEQ ID NO: 6 - DNA, 45 bp, Artificial Sequence
  Primer 080BN093 r
cannnnnnnnnnnnnnnnnnnnnnnattaatccctctgctccaac

SEQ ID NO: 7 - protein, 29 aa, Artificial Sequence
  BC2-Loop of the VP1-protein of the mouse polyomavirus strain BG with an integrated foreign epitope
YYGWSRGINXXXXXXXXXXXXNTLPTWSM

SEQ ID NO: 8 - DNA, 87 bp, Artificial Sequence
  Sequence coding for the BC2-Loop of the VP1- protein of the mouse polyomavirus strain BG with an integrated foreign epitope
tactatggttggagcagagggattaatnnnnnnnnnnnnnnnnnnnnnnnnnntggccannnnaatacacttcccacatggagtatg

SEQ ID NO: 9 - DNA, 87 bp, Artificial Sequence
  Counter strand of the sequence coding for the BC2-Loop of the VP1-protein of the mouse polyomavirus strain BG with an integrated foreign epitope
catactccatgtgggaagtgtattnnnntggccannnnnnnnnnnnnnnnnnnnnnnnnnattaatccctctgctccaaccatagta

(19) United States
(12) Patent Application Publication
(10) Pub. No.: US 2006/0222662 A1
(43) Pub. Date: Oct. 5, 2006

(54) Composition to be administered to a living being and method for marking agents

(75) Inventors: Jurgen Hess, Baiersdorf (DE); Markus Neugebauer, Erlangen (DE); Christoph Stark, Rheinfelden (DE); Sandra Strich, Erlangen (DE); Birgit Walders, Erlangen (DE)

Correspondence Address: KANESAKA BERNER AND PARTNERS LLP, SUITE 300, 1700 DIAGONAL RD, ALEXANDRIA, VA, 22314-2848, US

(73) Assignee: responsif GmbH, Erlangen (DE)

(21) Appl. No.: 10/546,031

(22) PCT Filed: Feb. 17, 2004
(86) PCT No.: PCT/EP04/01484

§ 371 Date: Oct. 7, 2005

(30) Foreign Application Priority Data
Feb. 18, 2003 (DE) 103 06 789.9

(51) Int. Cl. A61K 39/12; A61K 38/10; A61K 38/08

(52) U.S. Cl. 424/204.1; 514/13; 514/14; 514/15; 514/16; 514/17

(57) ABSTRACT

The invention relates to compositions to be administered to a living being, a method for marking agents that are administered to living beings, the use thereof, and an accelerated test.

The invention relates to a composition to be administered to a living being and to methods of labeling agents which are administered to living beings. The invention furthermore relates to uses of said composition and said method of the invention and to an accelerated test.

The labeling of substances is nowadays becoming more and more important. Whether it is fossil fuels to be labeled in order to be better able to monitor pollution possibly caused by said fossil fuel by detecting its origin, or whether it is the labeling of medicaments, for example vaccines, comprehensive detection of their origin with respect to both time and geography and also of their sale, their transport etc. is desired in all cases. A solid chain of detection is required in particular, for example, in the case of vaccines administered to humans and/or animals. In the past, this was attempted by labeling the corresponding packaging of the vaccine in a complicated manner. However, this “external labeling” has obvious disadvantages, since an unambiguous classification cannot be guaranteed, after the medicament, vaccine etc. have been administered or in the case of non-authorized replacement of the packaging, falsification of the label.

A, compared to this, more advantageous type of labeling is direct labeling of the medicament/vaccine etc. to be administered itself and not of its packing. This kind of “internal” labeling is proposed, for example, in DE 198 47 118, where in addition an immunogen which is harmless to the particular organism is admixed to the agent to be administered, which immunogen then elicits in said organism an immune response, in particular the formation of antibodies or T-cells. Proposed immunogens are: keyhole limpet hemocyanin (KLH) from Megathura crenulata, green fluorescent protein (GFP) from Aequoria victoria, inactive snake toxins and viral proteins. Advice on the use of virus-like particles cannot be found in DE 198 47 118. The immunogens disclosed in the prior art have the disadvantage that either they do not elicit any long-lasting immune responses after a single administration (e.g. KLH) and/or their preparation is clearly too expensive in order to be able to use them commercially on a larger scale (e.g. KLH or GFP). Owing to the time-limited traceability of the antibody response following a single KLH injection, for example, the non-responder rate in pigs increases dramatically during the second half of a fattening period (average fattening period in Germany: 20-24 weeks). Thus, there is a fundamental uncertainty in that it is not possible to detect subsequently, whether the animal/the patient to which whom the agent associated with KLH was administered simply did not exhibit any immune response (i.e. is a “non-responder”) or whether the agent/the vaccine was administered incorrectly (referred to as “non-compliance”).

Siray et al., 1998, Virus Genes 18, 39-47 disclose the possibility of expressing the VP1 protein of the polyoma virus from Syrian hamster (=golden hamster) in the form of an insoluble fusion protein in E. coli and the suitability of preparations of this kind for generating VP1 antisera in rabbits. However, the antiserum generated by Siray et al. showed crossreactivity to VP1 of other species, thus rendering impossible its use for labeling administered agents or agents to be administered.

Gedvilaite et al., 2000, Virology, 273, 21-35 describe the formation of Syrian hamster chimeric VLPs which are insoluble in aqueous solution and in which foreign epitopes have been incorporated. Gedvilaite et al. describe the use of these VLPs as vehicles for foreign vaccines incorporated in the form of epitopes into said VLPs. The study stresses the necessity of using complete virus-like particles (VLPs) in order to increase the epitope density and thereby to generate an immune response in the first place. The advantages of VLPs expressed in yeast are also noted, since these are the only endotoxin-free VLPs. In addition, the possibility of using Syrian hamster VLPs as possible carriers of gene constructs in gene therapy is mentioned.

It is the object of the present invention to provide a composition which, with respect to its labeling, can be prepared in a simple and inexpensive manner, which is absolutely harmless to the animal/the patient, which furthermore, with regard to its single label, produces in said animal/said patient a long-lasting immune titer higher or longer-lasting than the titer observed in connection with previous labels, and which, owing to its non-existing non-responder rate, renders a distinction between non-responder reaction and non-compliance unnecessary.

This object is achieved by a composition to be administered to a living being, comprising:

a) an agent selected from the group comprising medicaments, vaccines and stored blood, and

b) at least one type of protein complex, said protein complex being a single viral capsomer soluble in aqueous solution.

Preference is given to the protein complex being a single viral capsomer which is soluble in aqueous solution and which is an aggregated sandwich with other single viral capsomers soluble in aqueous solution.

In one embodiment, the protein complex is a monomeric viral capsomer.

In this connection, the term “monomeric” refers to the absence of an association with other viral capsomers.

In another embodiment, the protein complex is a viral capsomer which together with other viral capsomers forms an unspecific association of at least two capsomers. Said association varies in size as a function of the external conditions (e.g. buffer, temperature, concentration) and may comprise more than 20 capsomers. An association of this kind forms spontaneously under certain conditions and does not need any separate reconstitution step. An association of this kind is also referred to here as “aggregate” which, however, does not form a complete VLP or viral capsoid.

In one embodiment, the protein complex is not in the form of a viral capsoid. In one embodiment, the protein complex is soluble in aqueous solution.

Preference is given to the viral capsomer being produced recombinantly, particularly preferably in a prokaryotic expression system, in particular in E. coli.

In one embodiment, the viral capsomer is derived or can be obtained from a virus selected from the group of non-enveloped viruses, comprising Papovaviridae, in particular polyoma and papilloma viruses, Iridoviridae, Adenoviridae, Parvoviridae, Picornaviridae, in particular polio viruses, Caliciviridae, Reoviridae and Birnaviridae.

Preferably, the viral capsomer is derived or can be obtained from polyoma virus, in particular murine polyoma virus.

In one embodiment, the viral capsomer is a pentamer, hexamer or heptamer.

The terms “pentamer”, “hexamer”, “heptamer” refer to a single viral capsomer being composed of a plurality of viral capsomer proteins. Accordingly, a pentamer is a viral capsomer composed of five viral capsomer proteins, a hexamer is a viral capsomer composed of six viral capsomer proteins, etc.

In a preferred embodiment, the viral capsomer is a pentamer of murine polyoma virus VP1 or is a pentamer of murine polyoma virus VP1 in association with murine polyoma virus VP2, or is a pentamer of murine polyoma virus VP1 in association with murine polyoma virus VP3, or is a combination of the aforementioned possibilities. The term “pentamer . . . in association with VP2/3 . . . ” refers to the combination of a pentamer with a molecule VP2/3 . . . .

In an association of a VP1 pentamer with VP2, preference is given to VP2 being associated with at least one peptide. Preferably, the peptide is as defined below.

Particular preference is given to the viral capsomer being a pentamer of murine polyoma virus VP1.

In one embodiment, the viral capsomer is derived or can be obtained from a virus selected from the group of enveloped viruses, comprising Poxviridae, Herpesviridae, Hepadnaviridae, Retroviridae, Paramyxoviridae, Sendaiviridae, Orthomyxoviridae, Bunyaviridae, Arenaviridae, Toroviridae, Togaviridae, Flaviviridae, Rhabdoviridae and Filoviridae.

In one embodiment, the viral capsomer does not derive or cannot be obtained from a virus which enters the organism of the living being in the form of a vaccine or medicament or via the food chain or, under normal conditions of life of said living being, via the environment and/or to which antibodies are produced in said living being under normal conditions of life, it being preferred that the virus-like particle does not derive or cannot be obtained from a virus selected from the group comprising CSF virus (swine fever virus), foot-and-mouth disease virus, PPV (porcine parvovirus), influenza virus, in particular influenza A virus, bovine leukemia virus (EBL virus), bovine herpes virus (BHV1), bovine viral diarrhea virus (MD virus), bovine polyoma virus (BpyV), rotavirus, porcine herpes virus 1, pseudorabies virus, PRRS virus and TGE virus.

In one embodiment, the viral capsomer is associated with at least one peptide (association of viral capsomer and peptide).

Preference is given to the association of viral capsomer and peptide being soluble in aqueous solution.

In one embodiment, the peptide is immunogenic when administered to a living being, preference being given to said peptide being a peptide eliciting a B-cell response.

In one embodiment, the at least one peptide has been inserted recombinantly into the viral capsomers.

Preferably, the at least one peptide has a sequence derived from a virus, a prokaryotic cell or a eukaryotic cell. In one embodiment, the at least one peptide has a sequence which is of artificial origin.

In one embodiment, the peptide comprises no more than 5-35 amino acids, preferably no more than 5-20 amino acids and more preferably no more than 5-15 amino acids.

In one embodiment, the peptide is selected on the basis of one or more of the following criteria: probability of being located on the surface of a protein structure (surface probability), flexibility, hydropathy and antigenicity, with the peptide preferably having high surface probability, flexibility and antigenicity in conjunction with low hydropathy. When selecting the peptide in this way, one or more of the following methods may be applied, for example: [Boger, J., Emini, E. A. & Schmidt, A. Reports on the Sixth International Congress in Immunology (Toronto) 1986 p. 250; Chou P Y, Fasman G D. Adv Enzymol Relat Areas Mol Biol. 1978; 47:45-148; Emini E A, Hughes J V, Perlow D S, Boger J. J. Virol. 1985 September; 55(3): 836-9; Garnier, J. Osguthorpe, D. J. and Robson, B. J. Mol. Biol. 1978, 120, 97-120; Hirakawa H, Muta S, Kuhara S. Bioinformatic 1999 February; 15(2):141-8; Hopp T P, Woods K R. Proc Natl Acad Sci USA. 1981 June; 78(6): 3824-8; Jameson B A, Wolf H. Comput Appl Biosci. 1988 March; 4(1): 181-6; Janin J, Wodak S. J Mol Biol. 1978 Nov. 5; 125(3): 357-86; Kyte J, Doolittle R F. J Mol Biol. 1982 May 5; 157(1): 105-32; Parker J M, Guo D, Hodges R S, Biochemistry 1986 Sep. 23; 25(19): 5425-32; Welling G W, Weijer W J, van der Zee R, Welling-Wester S; FEBS Lett. 1985 Sep. 2; 188(2):215-8]. In one embodiment the peptide is determined via the metaepitopicity subprogram from Metalife AG (Metatope™, Metapark, 79297 Winden, Germany).

In one embodiment, the viral capsomer is derived from a first virus and the peptide is derived from a second virus which is not the same as the first virus.

In one embodiment, the peptide is derived or can be obtained from a virus selected from the group of non-enveloped viruses, comprising Papovaviridae, in particular polyoma and papilloma viruses, Iridoviridae, Adenoviridae, Parvoviridae, Picornaviridae, in particular polio viruses, Caliciviridae, Reoviridae and Birnaviridae.

In one embodiment, the peptide is derived or can be obtained from a virus selected from the group of enveloped viruses, comprising Poxviridae, Herpesviridae, Hepadnaviridae, Retroviridae, Paramyxoviridae, Sendaiviridae, Orthomyxoviridae, Bunyaviridae, Arenaviridae, Toroviridae, Togaviridae, Flaviviridae, Rhabdoviridae and Filoviridae.

In one embodiment, the peptide does not comprise any peptide epitope which is typically used for recording the infection status, disease status or vaccination status, for example in the course of vaccination programs.

An example of such a peptide epitope used for recording the infection status is the peptide epitopes of the non-structural protein (NSP) “3ABC”. This is a protein of the foot-and-mouth virus, which is not part of the viral envelope but to which an animal infected with the living virus produces antibodies regardless (in addition to the antibodies to the structural, i.e. envelope, proteins). In modern vaccines, however, such non-structural proteins of the foot-and-mouth disease virus have been removed so that an animal vaccinated with a vaccine prepared in this way does not produce any antibodies to the “3ABC” epitopes. If an infected animal or a vaccinated animal is tested for antibodies to “3ABC” epitopes by means of an immunochemical assay (for example ELISA), then the infected animal shows a positive response and the vaccinated animal shows a negative response. Consequently, “3ABC” or the epitopes present therein are used as differentiation markers when recording the infection status or vaccination status of animals. This enables vaccinated and infected animals to be distinguished from one another. An assay of this kind for “3ABC” is available from Intervet (http://www.intervet.com).

Preferably, the peptide does not derive or cannot be obtained from an agent, for example a virus, bacterium or a eukaryotic cell, which enters the organism of the living being in the form of a vaccine or medicament or via the food chain or, under normal conditions of life of said living being, via the environment and/or to which antibodies are produced in said living being under normal conditions of life, it being preferred that the peptide does not derive or cannot be obtained from a virus selected from the group comprising CSF virus (swine fever virus), foot-and-mouth disease virus, PPV (porcine parvovirus), influenza virus, in particular influenza A virus, bovine leukemia virus (EBL virus), bovine herpes virus (BHV1), bovine viral diarrhea virus (MD virus), bovine polyoma virus (BpyV), rotavirus, porcine herpes virus 1, pseudorabies virus, PRRS virus and TGE virus.

In one embodiment, the peptide does not derive from Leptospira, in particular L. grippotyphusa, L. tarassovi, L. canicola, L. pomona, L. bratislava, Chlamydia, in particular C. psittaci, Brucella, in particular B. abortus, B. canis, B. melitensis, Mycobacterium, in particular M. avium subsp. paratuberculosis or Coxiella, in particular C. burnetii.

In one embodiment, the peptide is an artificial peptide.

“Artificial” in this connection means that the peptide has a sequence which is of artificial origin, i.e. is a “fantasy sequence”. However, this should not rule out the possibility of finding in a database such an artificial sequence belonging to an organism. The only single criterion of such a sequence is the fact that it has been selected without taking into account or knowing its presence in a database.

In one embodiment, the at least one peptide has been coexpressed with the capsomer protein, starting from a DNA encoding said at least one peptide and said capsomer protein.

In one embodiment, the peptide is linked to the viral capsomer via a structure which mediates interaction, with the interaction-mediating structure preferably being located on the viral capsomer.

The interaction is preferably a hydrophobic interaction, a covalent bond, an ionic bond or a hydrogen bond between the viral capsomer and the at least one peptide.

In one embodiment, the structure which mediates interaction has preferably at least one bifunctional crosslinker, which is preferably a heterobifunctional crosslinker which particularly preferably has a moiety which is reactive to amino groups and a moiety which is different therefrom and which is reactive to sulfhydryl groups.

In one embodiment, the bifunctional crosslinker is selected from the group comprising maleimide derivatives, alkyl halides, aryl halides, isocyanates, glutardialdehydes, acrylating reagents and imido esters.

In one embodiment, the structure which mediates interaction has at least one affinity-increasing group which preferably is selected from the group comprising 4-iodoacetamidosalicylic acid, p-arsonic acid phenyldiazonium fluoroborate and derivatives thereof.

The viral capsomer is preferably associated with two or more peptides as defined above.

In this connection, the two or more peptides may have the same sequence or a different sequence. In the case of more than two peptides, these may have the same sequence or one or more different sequences.

In one embodiment, the viral capsomer and/or the at least one peptide are in the form of the nucleic acid coding therefor.

In one embodiment, the composition furthermore comprises an adjuvant. Preferably, the adjuvant is selected from the group comprising Montanide IMS 1312® and Quillaja Saponin (QuilA). Also suitable are CpG-DNA, aluminum adjuvants (e.g. aluminum hydroxide gels such as Alhydrogel), other saponins, aqueous adjuvants (such as, for example, Montanide IMS 1313®, Montanide IMS 1314®), water/oil emulsions, oil/water emulsions, water/oil/water emulsions (with metabolizable oils or with nonmetabolizable mineral oils), ISCOMs, liposomes, LPS and derivatives (such as, for example, MPL=Monophosphoryl Lipid A) as adjuvants.

Preferably, upon singular administration of said composition to a living being, the viral capsomer elicits in said living being an immune response which can still be detected at least 18 weeks, preferably at least 20 weeks, more preferably at least 24 weeks, post administration.

Preferably, the immune response manifests itself in the form of an increased anti-viral capsomer-IgG and/or -IgA titer and/or an increased anti-viral capsomer protein-IgG and/or -IgA titer and/or an increased anti-peptide-IgG and/or -IgA titer, it being preferred that the increased anti-viral capsomer/viral capsomer protein/peptide-IgG and/or -IgA titer is at least 1:64, more preferably at least 1:128, which, in one embodiment, is also still detectable at least 18 weeks, preferably at least 20 weeks, more preferably at least 24 weeks, post administration.

Detection is preferably carried out by means of an enzyme-immunological or immunochemical accelerated test or ELISA, which, in one embodiment, is performed on a body fluid selected from the group comprising meat juice, blood, whole blood, serum, plasma, lymph, urine, saliva, milk and semen.

The objects of the present invention are likewise achieved by a method of labeling agents administered to living beings, characterized by the following steps:

a) preparing a composition of the invention, as defined above, by adding a protein complex or viral capsomer as defined above to an agent to be labeled, as defined above,

b) administering said composition to a living being,

c) detecting the immunoresponse caused by said administration in said living being by means of an enzyme-immunological or immunochemical method.

Preference is given to the immune response comprising a formation of antibodies, said antibodies preferably being secreted antibodies and/or antibodies exposed on lymphocyte surfaces.

Preferably, detection takes place in a body fluid selected from the group comprising meat juice, blood, whole blood, plasma, lymph, serum, saliva, milk, urine and semen.

In one embodiment, the lymphocytes are B-lymphocytes and/or B-lymphocytes in combination with T-lymphocytes.

Preferably, the administration to a living being is carried out once or several times, in the latter case at intervals of several weeks, preferably 1-4 weeks.

In one embodiment, the agent is a medicament, a vaccine or stored blood, preferably an anti-infectious agent, in particular an antibiotic.

The objects of the present are also achieved by using the method of the invention and/or the composition of the invention for labeling living beings, said living being preferably being a non-human mammal, more preferably a mammal selected from the group comprising cattle, pigs, sheep, horses, hares, rabbits, dogs, cats, llamas, camels, marine mammals such as ceteaceans, seals and harbor seals.

The objects of the present invention are also achieved by using the method of the invention and/or the composition of the invention for immunological monitoring, it being preferred to check living beings or populations of living beings, as to whether they have come into contact with a particular agent, for example a vaccine, a medicament, a foodstuff, etc.

The objects of the present invention are also achieved by an antibody directed against the viral capsomer and/or against the at least one peptide of the composition of the invention.

The objects of the present invention are also achieved by an antibody directed against the aforementioned antibody.

The objects of the present invention are also achieved by an accelerated test comprising the last-mentioned antibody and/or the viral capsomer as defined above and/or the viral capsomer as described above in association with the at least one peptide as defined above.

Preference is given to the antibody and/or the viral capsomer being coupled to a reporter reagent.

Examples of the reporter reagent are colloidal gold, fluorescent dyes, biotin, alkaline phosphatase or peroxidase, preferably horseradish peroxidase. More preferably, the reporter reagent is colloidal gold.

The term “virus-like particle” (VLP), as used herein, refers to an agglomerate of viral proteins, which is incapable of replicating with the aid of the host cell metabolism, but which has the phenotype of a viral envelope, for example under an electron microscope. A virus-like particle, as used herein, represents a noninfectious viral envelope or parts thereof. The term “virus-like particle”, as used herein, is used synonymously with “capsoid”. The term “virus-like particle”, as used herein, is to be distinguished from the individual building blocks of a viral envelope, the “viral capsomers”. Thus, numerous viral envelopes consist of subunits, called capsomers, which make up the envelope. These “capsomers” in turn usually consist of one or more proteins, called viral capsomer proteins. The term “viral capsomer protein”, as used herein, refers to a subunit of a viral capsomer, it being possible for said viral capsomer to be composed of one or more viral capsomer protein molecules of one or more types.

The use “viral capsomer in association with other viral capsomers”, as used herein, refers to a combination of a variety of viral capsomers, for example of two or more viral capsomers. The term preferably refers to the combination of at least two viral capsomers. The term “association with other viral capsomers” may also include a complete viral capsoid. However, preference is given to an “association with other viral capsomers” not being a complete viral capsoid. The term “pentamer”, “hexamer” or “heptamer” when used in order to describe a viral capsomer in more detail, refer to a combination of five, six or sevel viral capsomer proteins, each of which result in a viral capsomer. The term “viral capsomer”, as used herein, is to be understood as meaning that the viral capsomer is not present in the form of a capsoid (or synonym: viral capsoid).

The use of viral capsomers in a composition to be administered has been shown to result in immune titers of antibodies/B-cells, which last distinctly longer or are distinctly higher than those which can be achieved by the immunogens used in the prior art (e.g. KLH, GFP, or else whole virus-like particles). The viral capsomers of the invention can be prepared recombinantly in a simple manner and exhibit no undesired side effects whatsoever in the organism to which the composition is administered.

In contrast to the use of complete virus-like particles, the immune response achieved when using viral capsomers is equally high or higher, without the complicated reconstitution step for preparing the viral capsoids or VLPs. As a result, the method of the invention is considerably less expensive. Moreover, in the case of the viral capsomers of the invention, an undesired crossreactivity can be ruled out, enabling viral capsomers only then to be used for labeling of agents to be administered or in a labeling process. The viral capsomers of the invention are soluble in aqueous solution and are therefore also suitable for the use in living animals, in particular farm animals.

In one embodiment of the present invention, the viral capsomers of murine polyoma virus have proved particularly advantageous. Murine polyoma virus has been linked with tumorigenesis in rodents. There are other species-specific or family-specific polyoma viruses, at least some of which have been found also to be tumorigenic, for example the primate virus SV40, bovine polyoma virus (BpyV), two human polyoma species (JC and BK), the two latter having been linked with progressive multifocal leukoencephalopathy (PML) and ureter stenosis in humans. Murine polyoma viruses is a double-stranded DNA virus belonging to the Papovaviridae family. The double-stranded DNA molecule consists of approximately 5000 bp and encodes five transcripts (T, t=early proteins, VP1, VP2 and VP3=late structural proteins). The viral envelope consists of three envelope proteins, VP1, VP2 and VP3, which may be used for the formation of virus-like particles (VLPs). However, the formation of VLPs does not require the presence of all three proteins. The isolated major envelope protein VP1 has been shown to form VLPs under particular conditions to be set by the person carrying out the experiment. The infectious murine polyoma virus has an envelope whose structure is formed by two shells, the outer shell of which consisting exclusively of VP1 and the inner shell of VP2 and VP3. It is therefore possible to generate noninfectious empty shells (virus-like particles) which consist exclusively of VP1. These empty envelopes may be assembled in vitro, for example using recombinant VP1, and are referred to, as used herein too, as “capsoids”, “viral capsoids” or “VLPs” or “virus-like particles” if they form a complete envelope. The VLPs are approximately 50 nm in diameter (as determined by means of electron microscopy) and are formed by 360 VP1 molecules which are arranged in 72 pentamers. Depending on the assembling conditions, however, it is also possible for smaller capsoids of 26 nm or 32 nm to be formed (Salunke et al., 1989, Biophys. J. 56 (5): 997-990). A VP1 pentamer is a “viral capsomer”, i.e. a capsoid-forming subunit. In this special case, a viral capsomer consists of five viral capsomer proteins, i.e. five VP1 molecules. The formation of pentamers (i.e. capsomer formation) is a process of spontaneous self-assembly which, in the case of recombinant expression of a VP1 protein in a host cell, for example E. coli, takes place immediately after expression, i.e. in vivo.

Reference is now made to the figures in which:

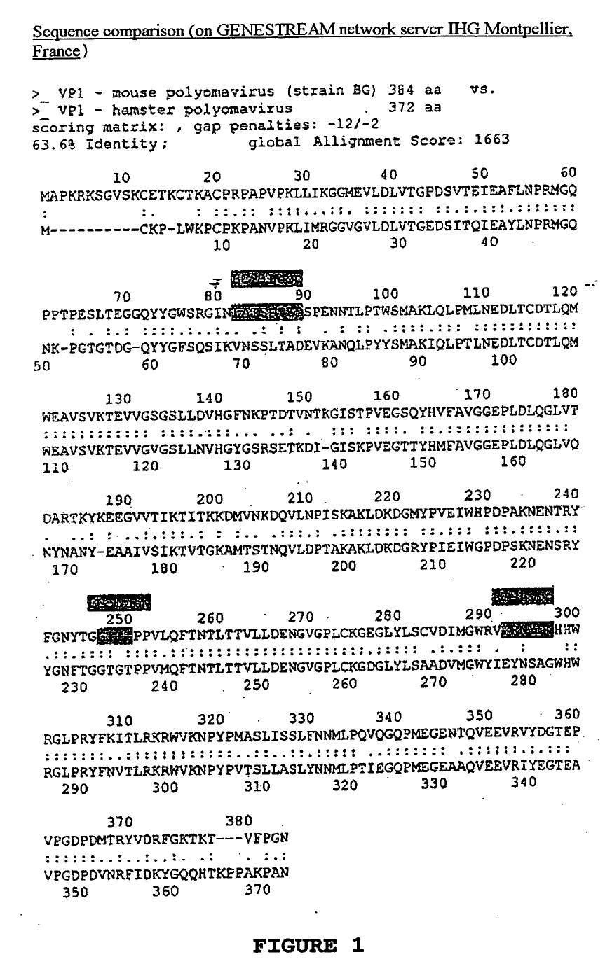

FIG. 1 depicts both a sequence comparison of the VP1 protein sequences of mouse polyoma virus (strain PG) and of hamster polyoma virus and possible sites of integration in outer loop regions of wild type VP1 (mouse polyoma virus),

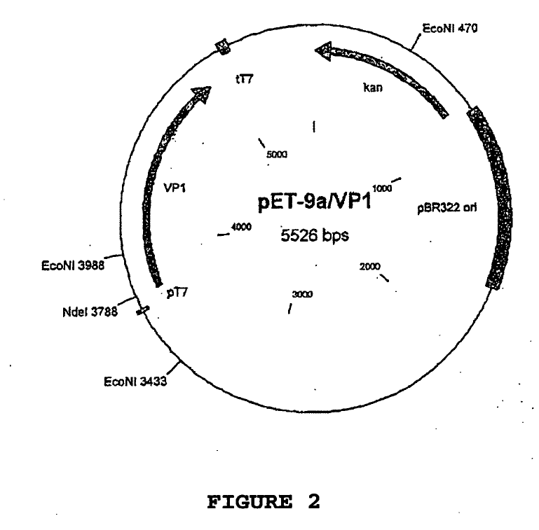

FIG. 2 depicts a VP1 expression vector (pET-9a/VP1),

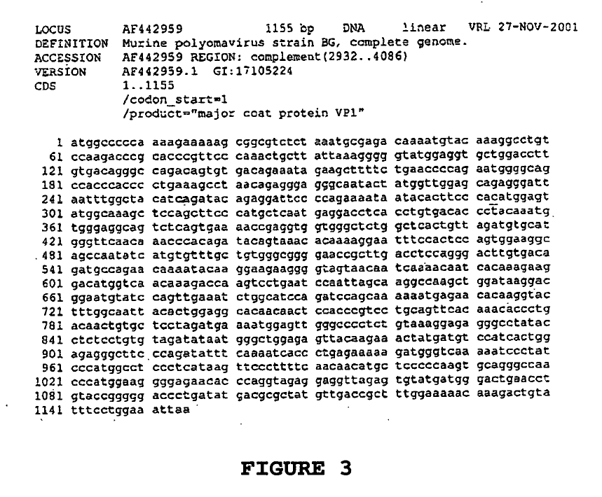

FIG. 3 depicts the VP1 protein-encoding DNA sequence of the mouse polyoma virus strain BG,

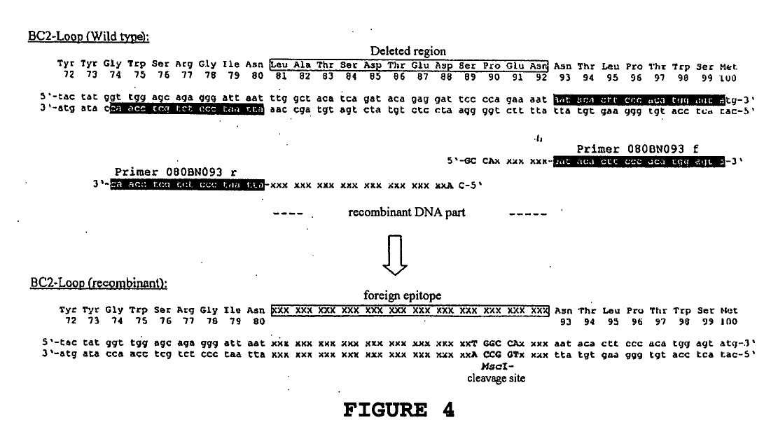

FIG. 4 depicts the integration of a foreign epitope into wild type VP1 by means of a PCR-based site-directed mutagenesis,

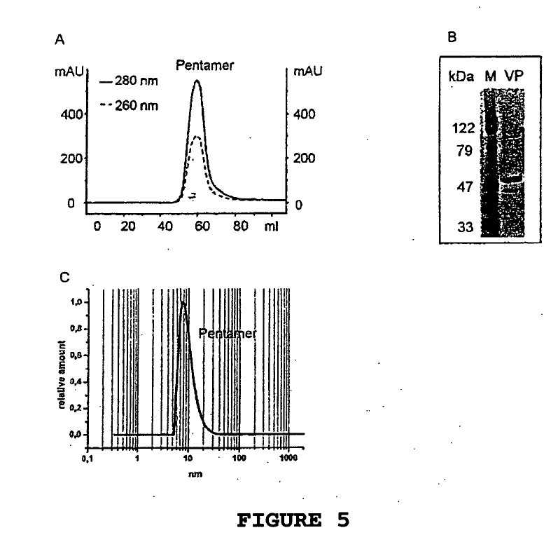

FIG. 5 depicts the purification of VP1-peptide capsomers, with FIG. 5A depicting a preparative gel filtration of the marker vaccine VP1-BC2, FIG. 5B depicting an SDS-PAGE analysis of the corresponding VP1 fraction, and FIG. 5C depicting a PCS (Photon Correlation Spectrometry) analysis of the corresponding VP1 fraction,

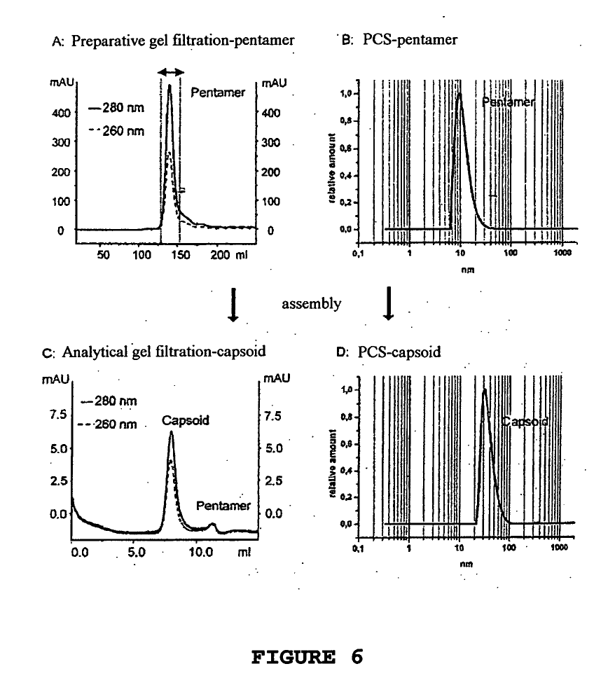

FIG. 6 depicts the assembly of VP1 capsomers to capsoids, with FIG. 6A depicting a gel filtration profile, FIG. 6B depicting the corresponding PCS measurement, FIG. 6C depicting the results of an analytical gel filtration and FIG. 6B depicting the results of a PCS measurement,

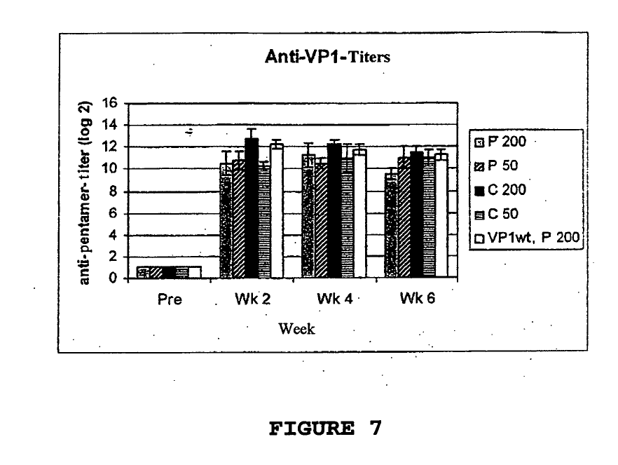

FIG. 7 depicts the time course of the immune responses in pigs, measured as anti-VP1 titer after immunization,

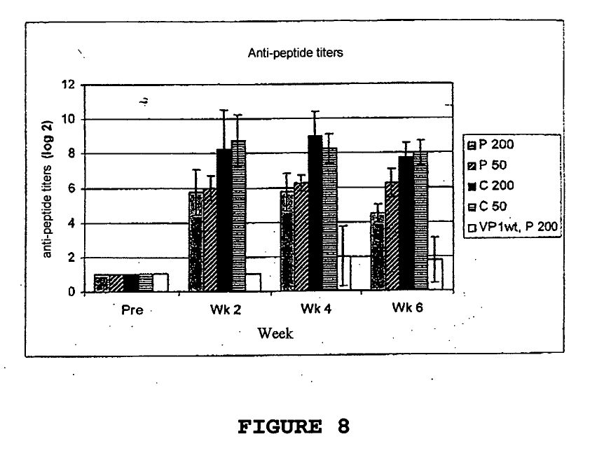

FIG. 8 depicts the time course of the immune responses in pigs, measured as anti-peptide titer after immunization,

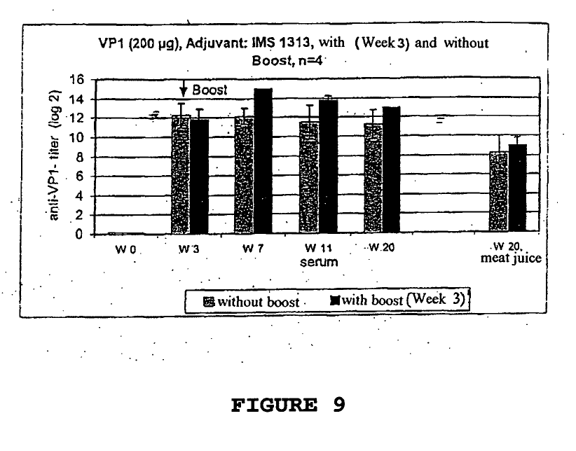

FIG. 9 depicts the time course of the immune responses in pigs after immunization with VP1 pentamers with and without boost immunization, measured as anti-VP1 titer over a period of 20 weeks,

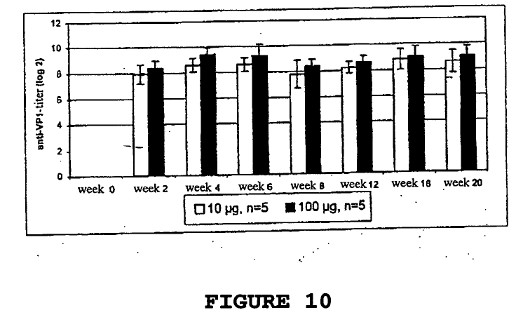

FIG. 10 depicts the time course of the immune responses in cattle to an immunization with different doses of viral capsoids over a period of 20 weeks,

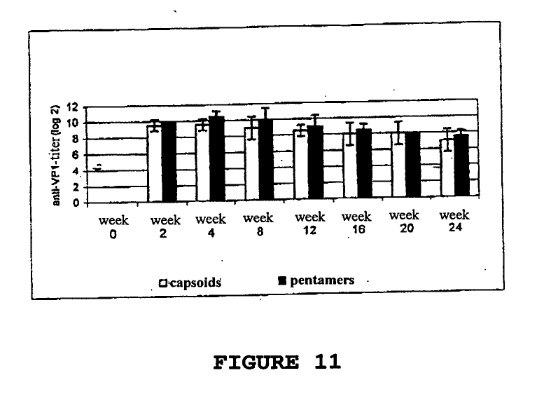

FIG. 11 depicts the time course of the immune responses in cattle to an immunization with VP1 capsoids and VP1 pentamers over a period of 24 weeks, and

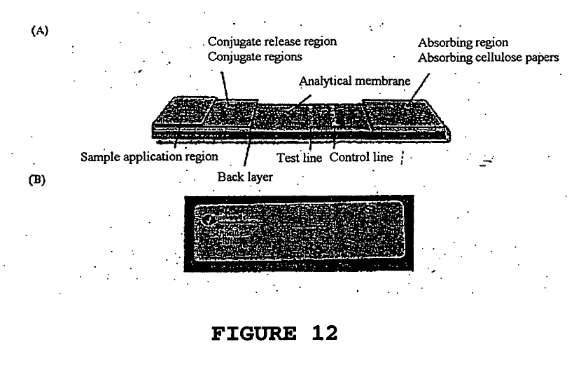

FIG. 12 depicts the diagrammatic structure of an embodiment of a corresponding accelerated test (12A) and a photographic view (12B) of an embodiment of such an accelerated test.

Reference is now made to the examples which are presented herein for the purpose of illustration and not by way of limitation.

### EXAMPLE OF VIRAL CAPSOMERS AS LABELING VACCINE

In this embodiment, soluble viral capsomers associated with a peptide sequence were used for labeling. The viral capsomers in this example consisted of pentamers of the murine polyoma virus envelope protein VP1. In addition, murine VP1 viral capsomers were employed in order to be able to compare the immune responses of capsomer and capsoid. The VP1 viral capsoids consist in each case of 72 capsomers (pentamers).

The peptide was selected according to the abovementioned definitions. In this embodiment, VP1 was linked to the peptide by directly cloning the peptide sequence into VP1. Piglets were selected as use examples. The immune response manifested itself here in the form of increased anti-VP1 and peptide-IgG titers, the antibodies were detected in the blood serum by means of ELISA.

### Example 1

### Binding of the Peptide to the Capsomer

In this embodiment, binding was effected by inserting the peptide sequence directly into murine VP1 polyoma virus.

For this purpose, surface-exposed, flexible regions within the VP1 structure were selected. The selection was carried out on the basis of the X-ray crystal structure of murine polyoma VP1 as pentameric asssemblage (1SID, PDB; according to Stehle and Harrison, 1996, Structure 4(2):183-94). The structural analysis with respect to the secondary, tertiary and quaternary structures was carried out with the aid of the VMD program (Virtual Molecular Dynamics) V.1.7.2 (Theoretical Biophysics Group, University of Illinois and Beckman Institute). In addition, the biochemical parameters: polarity, hydrophobicity and ionic interactions, were taken into account. Furthermore, insertion of the peptide should not influence the formation of pentamers.

On the basis of these criteria, the following regions (FIG. 1) were selected:
BC2 loop (sequence position 80-88)
HI loop (sequence position 291-296)
FG loop (sequence position 246-249)

(The loop regions were named according to Stehle and Harrison, 1996)

An extension of the strategy is the possibility of not only carrying out one peptide integration per VP1 monomer but also integrating simultaneously in different loop regions

a. the same epitope, in order to thereby increase the immunogen dose and thus the immune response;

b. various epitope sequences, in order to thereby modulate, different, specific immune responses.

The embodiment described below is the insertion of the peptide into the BC2 loop.

The study by Gedvilaite et al., 2000 (Virology 273(1): 21-35) involved inserting an epitope of hepatitis B virus into various regions of hamster polyoma VP1. However, since the sequence identity of hamster polyoma VP1 and murine polyoma VP1 is only 63.6% (FIG. 1), a direct comparison with this study is not possible.

FIG. 1 depicts, highlighted by a gray box, the core regions of the outer loop regions in wild type VP1, which were utilized for the integration of foreign sequences; moreover, the figure depicts a homology comparison of the VP1 protein sequences of mouse polyoma virus strain BG (in each case line 1) and hamster polyoma virus (in each case line 2).

### Example 2

### Cloning of the VP1 Mutants

Depending on the primary structure or length (8-20 amino acids) of the foreign epitope to be inserted and on the particular VP1 integration site, the loop regions of the wild type sequence were adjusted by “compensating deletions” of varying length (Δ=0 to 12 amino acids). On the one hand, it was intended to avoid disadvantageous structural alterations of the VP1 protein with effects on the solubility and assembly ability. On the other hand, it was intended to ensure good epitope exposition on the surface.

This adjustment was carried out with the aid of the 3D crystal structure image of wild type VP1 (Stehle and Harrison, 1996, EMBO J. 16(16): 5139-48 and Stehle and Harrison 1996, Structure 4(2): 165-82) and by means of suitable algorithms and structural predictions, using the Protean™ program (DNA-STAR Inc., Madison (Wis.), USA), according to:
Surface Probability—Emini et al., 1985, J. Virology 55: 836-9
Flexibility—Karplus and Schultz, 1965, Naturwissenschaften 72: 212-3
Hydropathy—Kyte and Doolittle, 1982, J. Mol. Biol. 157: 105-32
Hydropathy and Antigenicity—Hopp and Woods, 1981, Proc. Natl. Acad. Sci. 78: 3824-8
Antigenicity—Jameson and Wolf, 1968, CAPIOS 4: 181-6

In order to be better able to achieve the abovementioned criteria, it is possible to generate from the abovementioned types of integration further variants in which the peptide is inserted in the particular loop region with flanking, symmetrically arranged linkers. Said linker may be by way of example a tetrapeptide-serine/glycine linker ( . . . Ser-Gly-Ser-Gly-peptide-Gly-Ser-Gly-Ser . . . ). This peptide linker increases firstly the flexibility and secondly the hydrophobicity at the integration site. Other linkers having similar properties, such as serine/glycine dipeptide linkers, serine/glycine hexapeptide linkers, etc. and polyglycine linkers, are also conceivable for this purpose (Imanishi et al., 2000, Biochemistry 39(15): 4383-90 and Arakaki et al., 2002, Protein Expr Purif 25(2): 241-7).

2.1 Generation of the Expression Mutants

The integrations of the peptide-encoding foreign sequences and the deletions of the wild type sequences were generated by a PCR-based site-directe mutagenesis reaction directly with the entire, circular VP1 expression vector pET-9a/VP1 (5526 bp) (FIG. 2) as template. This rendered further subclonings unnecessary. The VP1 DNA (1155 bp) contained in the vector pET-9a (Novagen) is derived from the murine polyoma virus strain BG (GenBank/NCBI: accession number AF442959, FIG. 3) and is expressed as a protein of 384 amino acids without fusion tag under the control of a T7 promoter.

The VP1 expression vector pET-9a/VP1 depicted in FIG. 2 has the following molecular properties which can be found in the following table:

TABLE 1
Molecular properties:

| Name | Start | End | Description |
|---|---|---|---|
| Kann | 819 | 7 C | Kanamycin resistance |
| | | | gene |
| pBR322 ori | 1711 | 865 C | pBR322 origin of |
| | | | replication |
| pT7 | 3711 | 3727 | T7 promoter |
| VP1 | 3791 | 4945 | VP1-DNA |
| tT7 | 5077 | 5123 | T7 terminator |

The hybrid primer pairs used for mutagenesis contained at their 3′ ends adjacent wild type sequences which were complementary to the sense and antisense strands of the target DNA. In the case of deletions, the oligonucleotides hybridized with the wild type sequence at a greater distance from one another and thus left out a defined region.

The DNA sequence encoding the foreign epitope was distributed to the 5′ ends of the two hybrid primers and contained optimized codons for overexpression in E. coli (Shaun D. Black, University of Texas Health Center at Tyler; http://psyche.uthct.edu/shaun/SBlack/codonuse.html). The 5′ terminal positions were designed so as to contain in each case a portion of a unique recognition sequence for a restriction endonuclease (FIG. 4).

2.2. Example of a Mutagenesis in the Region Encoding the BC2 Loop

Unless stated otherwise, standard methods, for example according to Sambrook and Russell (Molecular Cloning, 5th ed., Cold Spring Harbor (N.Y.): Cold Spring Harbor Laboratory Press; c2001) were used.

The PCR mutagenesis was carried out using the oligonucleotides 080BN093 f and 080BN093 r (FIG. 4) and 100 ng of circular pET-9a/VP1 template DNA in 50 μl reaction mixtures according to standard methods. To reduce the error rate, PfuTurbo®DNA polymerase (Stratagene) with proofreading activity was used, only PAGE-purified oligonucleotides were utilized and the number of PCR cycles was limited to from 10 to no more than 18 cycles. The elongation was carried out at 68° C. for 2 minutes each per 1 kb. The PCR was carried out according to the following cycle profile:

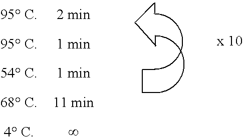

Part of the reaction mix was used for analyzing the linear vector product produced with respect to quantity and quality, in an agarose gel.

The remaining reaction mixture was incubated with 20 U of DpnI restriction endonuclease (New Enland Biolabs) at 37° C. for 1 h, whereby the methylated pET-9a/VP1 template DNA was selectively degraded.

After a “final polishing” with 1 U of PfuTurbo®DNA polymerase (Stratagene) at 68° C. for 0.5 h, in order to generate a complete set of blunt ends, the PCR products were isolated according to the E.Z.N.A.®Cycle-Pure protocol (PeqLab). The 5′ ends were phosphorylated by incubation with 5 U of T4 polynucleotide kinase (New England Biolabs) at 37° C. in a ligation buffer compatible therewith (New England Biolabs) for 0.5 h.

The linear amplification products were circularized by a 2 h short ligation at room temperature, using 400 NEB units (˜6 Weiss units) of T4 DNA ligase (New England Biolabs), and this reaction mixture was subsequently used for transforming the E. coli strain XL1 Blue (recA1 endA1 gyrA96 thi-1 hsdR17 supE44 relA1 lac [F′proAB lacIqZDM15 Tn10 (Tet^{r})]; Stratagene).

After selection on LB kanamycin agar, individual clones were amplified and the mini-prepared plasmid DNA (QIA-prep®Spin Miniprep Kit, Qiagen) was analyzed by restriction digest, in this case by MscI endonuclease in combination with NdeI endonuclease (New England Biolabs), with respect to the recombinant portion and the correct blunt-end fusion site. Recombinant plasmids were furthermore analyzed with regard to completeness of the plasmid by EcoNI restriction (3 cleavage sites in the vector sequence).

After fulfilling the two restriction criteria, the DNA of the VP1-encoding region was sequenced according to Sanger (Proc. Natl. Acad. Sci. USA (1977) 74, 5463-67) using the “ABI PRISM® Big Dye Terminators v 2.0 Cycle Sequencing Kit” (Applied Biosystems). In the case of an error-free sequence, the recombinant construct was transformed for expression into the E. coli strain BL21 (DE3) (F⁻ ompT hsdS_{B}(r_{B}⁻m_{B}⁻) gal dcm (DE3); Novagen).

FIG. 4 depicts the diagrammatic representation of the integration of a foreign epitope (12 amino acids) with a compensating deletion (12 amino acids) in the BC2 loop of wild type VP1 by a PCR-based site-directed mutagenesis.

### Example 3

### Preparation of Recombinant VP1-peptide Capsomers

In this embodiment, the VP1-peptide capsomer, referred to as VP1-BC2 hereinbelow, was purified recombinantly from E. coli.

When expressed in E.coli, the VP1 protein is in the form of pentamers (=capsomers). The assembly to capsomers must be induced, after purification of the pentamers, specifically by adding high salt concentrations (e.g. (NH₄)₂SO₄), oxidizing agents and Ca²⁺. In contrast, aggregates may form spontaneously depending on the purification and storage conditions of the capsomers. These aggregates are unspecific assemblages of at least two capsomers. The ratio of monomeric capsomers to capsomer aggregates and the size of the aggregates formed depend on the external conditions (see below). In this connection, the aggregates can be distinguished clearly from the capsoids: they do not form an envelope surrounding a cavity, but unspecific, irregular shapes. Aggregates may be the result of:
VP1-pentamer concentrations ≧1 mg/ml
vigorous or long-lasting shaking, vortexing, etc.
working at room temperature
long-term storage of the pentamers at temperatures ≧0° C.
no addition of reducing agents (e.g. DTT) and/or oxidation-inhibiting reagents (EDTA) to the buffer.

Depending on the buffer conditions, movements, temperature and type and duration of storage, the proportion of aggregates in the pentamer solution may be from 0 to above 50%. The size may also greatly vary, depending on the conditions (from approx. 2 to more than 20 pentamers). However, it is not possible to give an exact size, since the size depends on the external conditions, as listed above.

The aggregates may be detected by:
PCS measurement: While single VP1 capsomers have a specific size of 8-10 nm and capsoids have a size of 26 nm, 32 nm or 45 nm, aggregates have an unspecific size distribution of 20-100 nm, in extreme cases even more than 100 nm.
Gel filtration: Aggregates elute earlier than VP1 pentamers. Therefore, two separate signals (capsomer aggregates and monomeric capsomers) can be recognized in the gel filtration profile of VP1 pentamers in the case of aggregate formation. The proportion of aggregates can be quantified by gel filtration.
Electron microscopy: VP1 capsoids can clearly be recognized as envelopes containing a cavity, constructed from VP1 capsomers. In contrast, the aggregates can be seen as structureless, irregular capsomer assemblage. However, quantification of the aggregates (proportion/size) by electron microscopy is not possible.

The following example describes the protocol of the purification of VP1-BC2; the VP1 wild type was isolated according to the same principle.

After transformation of the VP1-BC2 plasmid into the E.coli strain BL21(DE3), expression, cell harvest and lysis, the protein was found to be in the soluble supernatant. Further isolation of the capsomers was carried out tag-free by a three-stage process.

After cation and anion exchanger, after-purification was carried out by way of a preparative gel filtration (HiLoad 16/60 Superdex 200 prep grade, Amersham Pharmacia) in PBS buffer. The gel filtration run in FIG. 5A shows only one peak at 55-65 ml. The SDS gel of the corresponding fraction shows VP1-BC2 (calculated mass=44 kDa) (FIG. 5B). Evaluation using the gel documentation system “ImageScanner” (Pharmacia) and the software “ImageMaster 1D, Version 4.00” (Pharmacia) reveals a purity of 75%. Checking the eluted VP1 fraction by photon correlation spectrometry (PCS) measurement using a high performance particle sizer (ALV-Sizer 2.9, ALV-NIBS) shows a particle size distribution of around 8 nm (FIG. 5C), which approximately corresponds to the size of 8-10 nm for VP1 wild-type capsomers, calculated by electron microscopy. Since neither the gel filtration profile nor the PCS measurement revealed additional protein or particle fractions, assembled capsoids or aggregates of capsomers may be present only to a small extent at this point in time.

After sterile filtration, the capsomers were used for labeling.

FIG. 5A depicts a preparative gel filtration of the marker vaccine VP1-BC2 over a HiLoad 16/60 Superdex 200 column. Only one VP1 fraction is detected. FIG. 5B depicts an SDS-PAGE analysis of the corresponding VP1 fraction. M=marker; VP=VP1 pentamer. FIG. 5C depicts the PCS analysis of the corresponding VP1 fraction.

### Example 4

### Assembly of the VP1 Capsomers

The assembly requires an aggregate-free capsomer fraction. In this case, the 3rd purification stage was carried out by way of preparative gel filtration (HiLoad 26/60 Superdex 200 prep grade, Amersham Pharmacia) in KB1 buffer (50 mM Na₂HPO₄, 150 mM NaCl, 2 mM EDTA, 5% glycerol, pH 6.8).

Here too (FIG. 6A), the gel filtration profile has only one VP1 peak (elution at 130-150 ml). Checking by PCS (photon correlation spectrometry) measurement indicates VP1 particles of around 10 nm in size (see FIG. 6B), corresponding to the abovementioned size of pentamers.

FIG. 6A depicts a preparative gel filtration of VP1-EC2 over a HiLoad 26/60 Superdex 200 column. Only one VP1 fraction is detected. FIG. 6B depicts the results of a PCS measurement of the correspponding VP1 fraction. FIG. 6C depicts the analytical gel filtration over a TSK Gel G 6000, PWXL column after assembly of the VP1 capsomers. The main elution fraction shows capsoids. FIG. 6D depicts a PCS analysis after assembly of the VP1 capsomers.

Subsequently, the capsomers were assembled to capsoids in an additional step according to standard methods (Stehle et al., 1994, Nature 369 (6476): 160-3 and Stehle et al., 1996, Structure 4(2): 165-82), which are known to the skilled worker. After the final dialysis, VP1-BC2 was in PBS+0.7 nM CaCl₂.

The size of average VP1 capsoids is indicated to be 45 nm, but smaller capsoids of 32 nm or 26 nm in size may also be produced, depending on the assembly conditions (Salunke et al., 1989, Biophys J. 56(5): 887-900). The PCS measurement carried out here following assembly indicated a particle size distribution of around 30-40 nm (FIG. 6D), approximately corresponding to the abovementioned data for capsoids. In addition, the assembly was checked by gel filtration using a TSK Gel G 6000-PWXL column (Toso Haas) (FIG. 6C). While the main elution fraction at 8 ml contains capsoids, non-assembled pentamers were present only in a smaller side fraction at 11 ml.

The VP1-BC2 capsoids were used for labeling, after sterile filtration.

### Example 5

### Analytical Testing of the Purified VP1 Mutants

5.1. Purity Analysis of the Protein by HPLC/mass Spectroscopy

HPLC/mass Spectroscopy

Besides SDS-gel analysis, the purity and exact mass were checked in this example via LC ESI-MS. The determination was carried out by means of the Agilent LC/MS 1100 Series and the “Agilent ChemStation, Version 08.03” software.

The VP1-BC2 mutant was, after reduction with DTT (dithiothreitol) fractionated by reversed phase HPLC with a gradient of 0-90% acetonitrile in H₂O containing 0.1% trifluoroacetic acid over a PLRP-S, 300 Å, 150×4.6 mm (Polymerlabs), and the protein was detected by measuring the absorption at 214 nm and 280 nm. The mass was determined directly thereafter by ESI-MS (electrospray ionization mass spectroscopy).

calculated mass of VP1-BC2: 44 334.2 Da. found mass of VP1-BC2: main mass: 44 316.6 Da (−17.4 Da).

In addition, an additional mass of 47 717.5 Da (+3401 Da) with a proportion of approx. 20% was found, which might indicate contaminations with E.coli proteins.

The data were recorded with an uncertainty interval of 12.39 Da and a standard deviation of 4.67 Da.

5.2. LPS Determination

This example utilized the LAL test (LAL=Limulus Amebocyte Lysate) by Charles River (Charleston, USA) for the determination of endotoxins. This test is based on the reaction of the LAL reagent with endotoxins, which proceeds with clouding and gel formation. The test was carried out according to the manufacturer's information following the kinetic turbidity method. The turbidity rate was measured by means of an ELISA reader (B Elx808 BIO-TEK reader) and evaluated using the “EndoScan V Software” (Charles River).

The result for the example of the VP1 mutant VP1-BC2 used here was an endotoxin concentration of:

185 EU/mg for VP1-BC2 pentamers

<42 EU/mg for VP1-BC2 capsoids

### Example 6

### Preparation of the Vaccine Doses for Pigs and Sterile Controls

In this example, in each case 50 μg or 200 μg of VP1-BC2 per vaccine dose were aliquoted under sterile conditions, admixed with sterile physiological saline (0.9% NaCl) to a volume of 1 ml and admixed with 1 ml of the adjuvant Montanide® IMS 1312 (Seppic, France). The finished vaccine solution was incubated with rolling at 4° C. overnight. Since the incubation was carried out with agitation and without addition of reducing agents, the capsomers could possibly have formed aggregates consisting of several pentamers, but this should not be regarded as fact. In contrast, the capsoids are extremely stable and cannot form any further aggregate forms.

For the sterile control, in each case 100 μl of the VP1 solution were removed, streaked onto CASO (Merck) agar plates and incubated at 37° C. After 24 h at 37° C., no colonies were found.

The vaccine doses for cattle were prepared analogously, but the adjuvant used was Quil A (1 mg/ml saline, Superfos, Denmark).

### Example 7

### Vaccination Schedule and Immunization

7.1. Vaccination Schedule

Pigs:

As an example, the immune response to the recombinant VP1-BC2 protein was first checked using piglets. The following problems were examined:
immune response to VP1-BC2 capsoids or pentamers
immune response to 50 μg or 200 μg of VP1-BC2

16 piglets (male and female) aged 19-21 days were selected and divided into 4 groups:

| Group | Vaccine dose | Adjuvant |
|---|---|---|
| Group 1 (4 animals): | 200 μg of VP1-BC2, pentamer | IMS 1312 |
| Group 2 (4 animals): | 50 μg of VP1-BC2, pentamer | IMS 1312 |
| Group 3 (4 animals): | 200 μg of VP1-BC2, capsoid | IMS 1312 |
| Group 4 (4 animals): | 50 μg of VP1-BC2, capsoid | IMS 1312 |

Cattle:

For dose comparison (capsoids 10 μg/dose or 100 μg/dose), in each case 5 cattle per dose were vaccinated; for a pentamer/capsoid comparison, in each case 2 cattle were vaccinated.

7.2. Immunization and Taking Blood

Pigs: The vaccination was carried out intramuscularly at the base of the ear. No group was boosted. Blood samples were taken from the vena cava cranialis on day 0 (preimmune serum), 14, 28 and 42.

Cattle: The vaccination was carried out subcutaneously on the side of the neck. The blood was taken from the external jugular vein on the day of immunization (preimmune sera) and after 2, 4, 6, 8, 12, 16, 20 weeks (dose comparison) or 2, 4, 8, 12, 16, 20, 24 weeks (pentamers/capsoids comparison).

### Example 8

### Detection of the Immune Response

In this example, the immune response manifested itself in the form of an increased anti-VP1 and anti-peptide-IgG titer. The titers were determined here by ELISA-testing the blood serum.

Pigs:

To detect the anti-peptide or -VP1 titers, the wells of an ELISA plate (C-MaxiSorp, Nunc) were coated with synthetic peptides of the corresponding sequence or with wild type-VP1 capsomers or corresponding capsoids. Incubation of the corresponding solutions at 4° C. overnight was followed by washing with PBS-T (PBS+0.5% Tween 20). Blocking was carried out with 1% BSA (bovine serum albumin, fraction V, Roth) in PBS, excess BSA was removed by washing with PBS-T. Subsequently, 100 μl of test serum, diluted in PBS-T with 0.5% BSA, negative and positive controls, were applied. The test serum was tested in decreasing concentration steps, with the serum concentration halved in each step (dilution 1:4; 1:8; 1:16 etc.). The samples were incubated on the plates for 1 h, excess reagent was removed by washing with PBS-T and affinity-purified biotin-coupled goat anti-pig IgG (Dianova) was added. After 3 more washing steps with PBS-T, the biotin-conjugated antibody was labeled by incubation with streptavidin-peroxidase (Roche). Excess reagent was removed with PBS-T. Development was carried out using the substrate 2,2′-azinobis(3-ethylbenzothiazoline-6-sulfonic acid) diammonium salt (Roche) which reacts with peroxidase to give a green color. The reaction was stopped by adding oxalic acid. The quantitative evaluation was carried out by means of an ELISA reader (Multiscan Ascent, Labsystems) by determining the optical density at 405 nm with 492 nm as reference (OD_{405/492 nm}).

Cattle:

To detect the anti-VP1 titers, the wells of an ELISA plate (C-MaxiSorp, Nunc) were coated with wild type-VP1 capsomers or corresponding capsoids. Incubation of the corresponding solutions at 4° C. overnight was followed by washing with PBS. Blocking was carried out with 1% BSA (bovine serum albumin, fraction V, Roth) in PBS, excess BSA was removed by washing with PBS-T (PBS+0.05% Tween 20). Subsequently 50 μl of test serum, diluted in PBS-T with 50 mM EDTA, negative and positive controls, were applied. The test serum was tested in decreasing concentration steps, with the serum concentration being halved in each step (dilution 1:4; 1:8; 1:16 etc.). The samples were incubated on the plates for 1 h, excess reagent was removed by washing with PBS-T and affinity-purified goat anti-bovine IgG peroxidase conjugate (Dianova) was added. Excess reagent was removed by PBS-T. The development was carried out using the substrate 2,2′-azinobis(3-ethylbenzothiazoline-6-sulfonic acid) diammonium salt (Roche) which reacts with the peroxidase to give a green color. The reaction was stopped by adding oxalic acid. The quantitative evaluation was carried out by means of an ELISA reader (Multiscan Ascent, Labsystems) by determining the optical density at 405 nm, with 492 nm as reference (OD_{405/492 nm}).

To calculate the antibody titers, first the cut-off value was determined as follows:

cut-off=average of OD_{405/492 nm} of the negative control+3*standard deviation.

A dilution of the test serum is denoted positive, if the OD_{405/492 nm} is higher than the cut-off value. The antibody titers are obtained by calculating the log 2 of the reciprocal value of the highest positive dilution of the test serum, i.e. an anti-VP1 titer or anti-peptide titer of 8 means that antibodies could still be detected at a dilution of the test serum of 1:2⁸=1:256.

### Example 9

### Time Course of the Immune Response in Piglents

9.1. Time Courses of the Anti-VP1 Titers

FIG. 7 depicts the time course of the immune responses to the VP1-BC2 capsomer and VP1-BC2 capsoid, selected here by way of example, i.e. depicts the time course of the anti-VP1 titers after immunization with: 200 μg of VP1-BC2 pentamer (P200); 50 μg of VP1-BC2 pentamer (P50), 200 μg of VP1-BC2 capsoid (C200); 50 μg of VP1-BC2 capsoid (C50); 200 μg of VP1 wild type pentamer (VP1wt, P 200). The average and standard deviation of each group are indicated (n=4). For each measurement (Pre=before immunization, Wk 2=week 2, Wk 4=week 4, . . . ), the corresponding immunizations are plotted as bars from left to right, i.e. the first bar from the left is P200, the second bar from the left is P50, etc.

For detection of the anti-VP1 titers, the test was carried out only against wild type-VP1 pentamers, and therefore peptide-specific antibodies are not included. Anti-VP1-IgG titers of ≦5 were regarded as negative and were given a score of log 2 titer level 1. While preimmune sera were, without exception, anti-VP1 negative, all animals were evaluated as anti-VP1 positive 2-6 weeks after labeling. VP1-BC2 induced high immune responses (log 2 titers of 10-12), and even 6 weeks after immunization there was still no distinct decrease in the immune response recordable. Significant differences were found neither between viral capsomers and capsoids nor between vaccine doses of 200 μg and 50 μg, meaning that it is possible to use viral capsomers for labeling without problems, i.e. the complicated reconstitution/assembly step is dispensed with.

9.2. Time Courses of the Anti-peptide Titers

FIG. 8 depicts the time course of the immune responses to the peptide inserted into VP1 by way of example, i.e. depicts the time course of the anti-peptide titer after immunization with: 200 μg of VP1-BC2 pentamer (P200); 50 μg of VP1-BC2 pentamer (P50), and 200 μg of VP1-BC2 capsoid (C200); 50 μg of VP1-BC2 capsoid (C50); and 200 μg of VP1 wild type pentamer (VP1wt, P 200). The average and standard deviation of each group are indicated (n=4). For each measurement (Wk 2=week 2, Wk 4=week 4, Pre=before immunization), the corresponding immunizations are plotted as bars from left to right, i.e. the first bar on the left is P200, the second bar from the left is P50, etc.

For detection of the anti-peptide titers, tests were only carried out against the peptide. Anti-peptide-IgG titers of □ 3 were regarded as negative and given a score of the log 2 titer level of 1. As expected, both the preimmune sera and the animals labeled with VP1 wild type had anti-peptide-negative immune responses (exception: in each case 1 animal of the VP1 wild type group in weeks 4 and 6). In contrast, all groups vaccinated with VP1-BC2 were registered as anti-peptide positive 2-6 weeks after labeling. The antibodies specifically directed against the peptide reached log 2 titer levels of up to 9 as a group average (200 μg of capsoid, week 4). In contrast to the capsoids, the capsomers had anti-peptide titers which were lower by 2-3 titer levels, but they are still detectable as positive in the ELISA. Reducing the dose here seems to result in an increase in the immune response.

Overall, the values of the anti-peptide titers are below those of the anti-VP1 titers. Since the total surface of the peptide is distinctly smaller than that of the carrier capsomer and since the carrier capsomer has a larger number of different epitopes, such a difference was to be expected.

9.3 Time Courses of the Anti-VP1 Titers—Comparison: Pentamers with and without Boost

FIG. 9 depicts the time course of the immune responses to VP1 capsomers in pigs over a longer period (W0, W3, W7 etc.=week 0, week 3, week 7 etc.). The time course of the anti-VP1 titers in pigs after immunization with in each case 200 μg of VP1 capsomers with and without boost (in week 3) is shown. The antigen-specific detection of antibodies was carried out by means of ELISA on microtiter plates coated with VP1 pentamers. In addition, the adjuvant Montanide IMS1313 was used in the immunization. The average and standard deviation of each group are indicated (n=4). For detection of the anti-VP1 titers, only tests against wild type-VP1 were carried out. Anti-VP1-IgG titers of ≦5 were regarded as negative. It is revealed that the groups without boost immunization also have a long-lasting immune response. Without boost immunization, a titer reduction by only 1 titer level (log 2 value) is recorded within a period of 20 weeks. In both animal groups, anti-VP1 antibody titers of 11-13 (log 2 value) are achieved up to week 20. Even in the meat juice, the label is still clearly detectable in both groups.

The data illustrate that long-term labeling with the composition of the invention is also possible in pigs.

In summary, this example indicates that labeling based on immunogenicity by administrating viral capsomers is a simple and cost-effective method. The viral capsomers used here elicit a high anti-VP1 immune response. No significant difference with respect to the anti-VP1 titers was found when comparing between the vaccination with capsoids and pentamers. Thus the costly and complicated step of assembly to capsoids is no longer necessary.

### Example 10

### Long-term Time Course of the Immune Response in Cattle

10.1 Time Course of the Anti-VP1 Titers—Dose Comparison

FIG. 10 depicts the time course of the immune responses to polyoma VP1 capsoids, i.e. the time course of the anti-VP1 titers after immunization with: 10 μg or 100 μg of polyoma VP1 capsoid in the presence of the adjuvant Quil A. The average and standard deviation of each group are indicated (n=5). For detection of the anti-VP1 titers, tests were only carried out against wild type VP1; therefore, peptide-specific antibodies are not included. Anti-VP1-IgG titers of ≦5 were regarded as negatives. All animals were evaluated as anti-VP1 positive as early as in week 2 after labeling. The polyoma VP1 capsoid-induced immune responses were in the range from 8-10, and remained up to week 20 after immunization. Significant differences with regard to the two concentrations used were not found. Detection of the antigen-specific antibodies in serum-immunized cattle was carried out by means of ELISA on microtiter plates coated with VP1 pentamers.

10.2 Time Courses of the Anti-VP1 Titers—Comparison: Pentamers—Capsoids

FIG. 11 depicts the time course of the immune responses to VP1 capsoids and VP1 capsomers in cattle over a relatively long period. The time course of the anti-VP1 titers after immunization with in each case 100 μg of VP1 capsoid or 200 μg of pentamers (=capsomers) in cattle is depicted there. The antigen-specific detection of antibodies was carried out by means of ELISA on microtiter plates coated with pentamers and capsoids, respectively. In addition, the adjuvant Quil A was used in the immunization. The average and standard deviation of each group are indicated (n=2). For detection of the anti-VP1 titers, only tests against wild type VP1 were carried out. Anti-VP1-IgG titers of ≦5 were regarded as negative. It is revealed, that the pentamers elicit identical or higher immune responses than the fully assembled VP2 capsoids. These responses remain even in week 24, illustrating that the composition of the invention or the method of the invention have excellent suitability for long-term labeling.

### Example 11

### Accelerated Test

FIG. 12 depicts an exemplary embodiment of an accelerated test of the invention which is suitable for detecting the label(s) in situ. The antigen (i.e. the viral capsomer or the peptide coupled to bovine serum albumin [BSA]) is immobilized on an analytical membrane on the test line (T). Downstream of the analytical membrane is a conjugate release region which contains a dried conjugate of gold with antibodies which are specific for IgG or IgA of the living being to be tested (i.e. cattle, pigs, etc.). Furthermore, the accelerated test has a sample application region. The use is as follows: the liquid sample containing the body fluid to be examined in which a possible immune response is to be tested is applied to the sample application region and, owing to capillary forces, migrates through the conjugate region, whereby the gold conjugate is rehydratized, enabling an interaction between the anti-peptide antibodies or anti-viral capsomer antibodies present in the body fluid and the gold conjugate, if such antibodies are present in the body fluid. The complex of gold and the two antibodies migrates to the test line at which the antigen is located (antigen coupled to BSA or another carrier), and is immobilized there and generates a colored line. A second line, the “control line”, indicates in each case of a correctly carried out test a signal which is mediated via a biotin-streptavidin binding. Biotin is located on the membrane and streptavidin is located in the gold conjugate.

The staining of the control line indicates that the accelerated test is completed. This test may provide rapid results within 5 minutes. Similarly, “dip sticks” may be constructed which are based on the same principle and in which a sample contact region is contacted with the body fluid to be studied and in which the subsequent reactions proceed in the same manner as in the accelerated test just described.

The previous examples of labeling with capsomers have shown that long-term labeling over 24 weeks is possible without boost, i.e. without further administration of the antigen. The stability of the anti-capsomer titers is thus extremely high.

By linking immunogenic peptide sequences to the carrier capsomer, a multiplicity of labeling combinations may be used. The direct cloning of the peptide sequence(s) into the capsomer as illustrated in Example 2, dispenses with the additional complicated step of conjugating the peptide/peptides to the capsomer.

The use of a simple accelerated test as described in Example 11, makes using the composition of the invention or the method of the invention extremely simple and uncomplicated and thus is a simple way which can be used even by non-experts to carry out the appropriate labeling tests or the appropriate monitoring.

The features disclosed in the above description, the claims and the drawings may, both individually and in any combination, be important for implementing the invention.

1-46. (canceled)

47. The use of a protein complex for preparing a composition for labeling living beings, the protein complex being a single viral capsomer which is not in the form of a viral capsoid and is soluble in aqueous solution, the viral capsomer being produced recombinantly and being associated with at least one peptide which is immunogenic when administered to a living being, the at least one peptide having been inserted recombinantly into the viral capsomer,
the viral capsomer being derived from a virus selected from the group of non-enveloped viruses, comprising Papovaviridae, Iridoviridae, Adenoviridae, Parvoviridae, Picomaviridae, Caliciviridae, Reoviridae and Bimaviridae.

48. The use as claimed in claim 47, wherein the protein complex is a single viral capsomer which is soluble in aqueous solution and which is an aggregated sandwich with other single viral capsomers soluble in aqueous solution.

49. The use as claimed in claim 47, wherein the Papovaviridae comprise polyoma and papilloma viruses and the Picomaviridae comprise polio viruses.

50. The use as claimed in claim 47, wherein the viral capsomer is derived from polyoma virus, in particular murine polyoma virus.

51. The use as claimed in claim 47, wherein the viral capsomer is a pentamer, hexamer or heptamer.

52. The use as claimed in claim 50, wherein the viral capsomer is a pentamer of murine polyoma virus VP1 or is a pentamer of murine polyoma virus VP1 in association with murine polyoma virus VP2, or is a pentamer of murine polyoma virus VP1 in association with murine polyoma virus VP3, or is a combination of the aforementioned possibilities.

53. The use as claimed in claim 52, wherein the viral capsomer is a pentamer of murine polyoma virus VP1.

54. The use as claimed in claim 47, wherein the viral capsomer does not derive or cannot be obtained from a virus selected from the group comprising CSF virus (swine fever virus), foot-and-mouth disease virus, PPV (porcine parvovirus), influenza virus, in particular influenza A virus, bovine leukemia virus (EBL virus) (BLV), bovine herpes virus (BHV1), bovine viral diarrhea virus (MD virus), bovine polyoma virus (BpyV), rotavirus, porcine herpes virus 1, pseudorabies virus, PRRS virus and TGE virus.

55. The use as claimed in claim 47, wherein the association of viral capsomer and peptide is soluble in aqueous solution.

56. The use as claimed in claim 47, wherein the peptide is a peptide eliciting a B-cell response.

57. The use as claimed in claim 47, wherein the peptide has a sequence derived from a virus, a prokaryotic cell or a eukaryotic cell or that the peptide has a sequence which is of artificial origin.

58. The use as claimed in claim 47, wherein the peptide comprises no more than 5-35 amino acids.

59. The use as claimed in claim 58, wherein the peptide comprises no more than 5-20 amino acids.

60. The use as claimed in claim 59, wherein that the peptide comprises no more than 5-15 amino acids.

61. The use as claimed in claim 47, wherein the viral capsomer is derived from a first virus and the peptide is derived from a second virus which is not the same as the first virus.

62. The use as claimed in claim 61, wherein the peptide is derived or can be obtained from a virus selected from the group of non-enveloped viruses, comprising Papovaviridae, in particular polyoma and papilloma viruses, Iridoviridae, Adenoviridae, Parvoviridae, Picomaviridae, in particular polio viruses, Caliciviridae, Reoviridae and Bimaviridae.

63. The use as claimed in claim 62, wherein the peptide is derived or can be obtained from a virus selected from the group of enveloped viruses, comprising Poxviridae, Herpesviridae, Hepadnaviridae, Retroviridae, Paramyxoviridae, Sendaiviridae, Orthomyxoviridae, Bunyaviridae, Arenaviridae, Toroviridae, Togaviridae, Flaviviridae, Rhabdoviridae and Filoviridae.

64. The use as claimed in claim 47, wherein the peptide does not derive or cannot be obtained from an agent, for example a virus, bacterium or a eukaryotic cell, which enters the organism of the living being in the form of a vaccine or medicarnent or via the food chain or, under normal conditions of life of said living being, via the environment and/or to which antibodies are produced in said living being under normal conditions of life.

65. The use as claimed in claim 64, wherein the peptide does not derive or cannot be obtained from a virus selected from the group comprising CSF virus (swine fever virus), foot-and-mouth disease virus, PPV (porcine parvovirus), influenza virus, in particular influenza A virus, bovine leukemia virus (EBL virus) (BLV), bovine herpes virus (BHV1), bovine viral diarrhea virus (MD virus), bovine polyoma virus (BpyV), rotavirus, porcine herpes virus 1, pseudorabies virus, PRRS virus and TGE virus.

66. The use as claimed in claim 65, wherein the peptide does not derive from Leptospira, in particular L. grippotyphusa, L. tarassovi, L. canicola, L. pomona, L. bratislava, Chlamydia, in particular C. psittaci, Brucella, in particular B. abortus, B. canis, B. melitensis, Mycobacterium, in particular M. avium subsp. paratuberculosis or Coxiella, in particular C. burnetii.

67. The use as claimed in claim 47, wherein the peptide is an artificial peptide.

68. The use as claimed in claim 47, wherein the at least one peptide has been coexpressed with the capsomer protein, starting from a DNA encoding said at least one peptide and said capsomer protein.

69. The use as claimed in claim 47, wherein the viral capsomer is associated with two or more peptides as defined in any of the preceding claims.

70. The use as claimed in claim 47, wherein the viral capsomer and/or the at least one peptide are in the form of the nucleic acid coding therefor.

71. The use as claimed in claim 47, wherein, upon singular administration oi said composition to a living being, the viral capsomer elicits in said living being an immune response which can still be detected at least 18 weeks post administration.

72. The use as claimed in claim 71, wherein the immune response can still be detected after at least 20 weeks.

73. The use as claimed in claim 72, wherein the immune response can still be detected at least 24 weeks post administration.

74. The use as claimed in claim 71, wherein the immune response manifests itself in the form of an increased anti-viral capsomer-IgG and/or -IgA titer and/or an increased anti-viral capsomer protein-IgG and/or -IgA titer and/or an increased anti-peptide-IgG and/or -IgA titer.

75. The use as claimed in claim 74, wherein the increased anti-viral capsomer/viral capsomer protein/peptide-IgG and/or -IgA titer is at least 1:64.

76. A method of labeling living beings or agents administered to living beings, comprising the following steps:
a) adding a protein complex as defined in claim 47 to an agent to be labeled,
b) administering said agent to a living being,
c) detecting the immunoresponse caused by said administration in said living being by means of an enzyme-immunological or immunochemical method.

77. The method as claimed in claim 76, wherein the immune response comprises a formation of antibodies.

78. The method as claimed in claim 77, wherein the antibodies are secreted antibodies and/or antibodies exposed on lymphocyte surfaces.

79. The method as claimed in claim 76, wherein detection takes place in a body fluid selected from the group comprising meat juice, blood, whole blood, plasma, lymph, serum, saliva, milk, urine and semen.

80. The method as claimed in claim 78, wherein the lymphocytes are B-lymphocytes and/or B-lymphocytes in combination with T-lymphocytes.

81. The method as claimed in claim 76, wherein the administration is carried out once or several times, in the latter case at intervals of several weeks.

82. The method as claimed in claim 76, wherein agent is a medicament, a vaccine or stored blood.

83. The method as claimed in claim 82, wherein the agent is an anti-infectious agent, in particular an antibiotic.

84. The method as claimed in claim 76, wherein the living being is a non-human mammal.

85. An antibody directed against the viral capsomer and/or the at least one peptide of the protein complex as defined in claim 47.

86. An antibody directed against the antibody as claimed in claim 85.

87. An accelerated test comprising the antibody as claimed in claim 86 at least one of the viral capsomer as defined in claim 47 and/or the peptide as defined in claim 47.

88. The accelerated test as claimed in claim 87, wherein the antibody and/or the viral capsomer and/or the peptide are coupled to a reporter reagent.
